(19) United States
(12) Patent Application Publication
(10) Pub. No.: US 2026/0249006 A1
(43) Pub. Date: Aug. 27, 2026

(54) MEDICATION DELIVERY DETECTOR

(71) Applicant: BECTON, DICKINSON AND COMPANY, Franklin Lakes, NJ (US)

(72) Inventors: Yiping MA, Layton, UT (US); Alexandra DOWNS, Ridgewood, NJ (US)

(21) Appl. No.: 19/540,934

(22) Filed: Feb. 16, 2026

Related U.S. Application Data
(60) Provisional application No. 63/764,299, filed on Feb. 27, 2025.

(51) Int. Cl. A61M 5/172; A61M 5/142; A61M 5/168; G16H 20/13; G16H 20/17

(52) U.S. Cl. CPC A61M 5/1723; A61M 5/14244; A61M 5/16831; G16H 20/13; G16H 20/17

(57) ABSTRACT

A medication delivery detector includes a body having a proximal end and a distal end, a first connector coupled to the proximal end of the body, a second connector coupled to the distal end of the body, and a lumen formed in the interior of the body. At least one sensor is exposed to the lumen to detect an identity and concentration of a fluid substance flowing through the lumen, wherein the at least one sensor comprises a receptor and sensor electrodes.

This application claims the benefit of U.S. Provisional Patent Application Serial No. 63/764,299, filed February 27, 2025, the disclosure of which is incorporated herein by reference.

### TECHNICAL FIELD

The present disclosure generally relates to devices and methods for detecting the delivery of medication to a patient. In particular, the present disclosure relates to medication delivery devices and methods that utilize sensors to capture the presence and concentration of various medications.

### BACKGROUND

The administration of medications to patients is continuously monitored by hospitals and other healthcare facilities during treatment of an individual, as accurate dosing of medication may ensure a more safe and effective treatment. Errors in the administration of medicine can lead to overdose, missed doses, treatment failure, and other adverse health outcomes. Despite enhanced methods for tracking medication, errors in the administration of medicine remain prevalent, particularly in clinical settings.

Current devices and methods for administering medication often rely on manual recording, barcode scanning, or radio frequency identification technology to track medication from the preparation stage to bedside delivery. However, these devices and methods allow for shortfalls caused by human error or a lack of real-time monitoring capabilities. For example, even when a device detects a deviation from the prescribed dosage, the device fails to provide comprehensive oversight by shutting off the delivery device and alerting healthcare providers.

Accordingly, a need exists for a device capable of detecting a type and dosage of medication that is being delivered to a patient and providing actionable feedback to clinicians. There is demand for a device configured to improve the efficiency and accuracy of medication administration, thus reducing the risk of error and contributing to better patient outcomes.

### SUMMARY

According to one aspect of the disclosure, a medication delivery detector is provided. The detector comprises a body having a proximal end and a distal end. A first connector is coupled to the proximal end of the body, and second connector coupled to the distal end of the body. A lumen is formed in the interior of the body, and at least one sensor is exposed to the lumen to detect an identity and concentration of a fluid substance flowing through the lumen, wherein the at least one sensor comprises a receptor and sensor electrodes.

In some embodiments, a valve is integrated into the lumen of the body, whereby the fluid substance flows through the valve. The valve alternates between an open position and a closed position. The valve may be located between the at least one sensor and the second connector.

In some embodiments, the at least one sensor comprises a redox active receptor. The at least one sensor may include a plurality of sensors, which may be spaced along a length of the body. The plurality of sensors may be disposed throughout the body extend circumferentially about the lumen formed in the interior of the body. The plurality of sensors disposed may be throughout the body on a first side of the lumen are staggered in relation to the plurality of sensors on a second side of the lumen.

In some embodiments, at least one flow rate sensor is integrated into the body. In such case, the at least one sensor is integrated into the body between a first flow rate sensor on the proximal end and a second flow rate sensor on the distal end. The at least one flow rate sensor may be integrated into the body between the at least one sensor and a valve. The valve may be integrated into the body between the at least one flow rate sensor and the second connector on the distal end.

The detector may be configured to interface with a catheter. The detector may be configured to interface with an infusion device. A control unit may be coupled to the body, which unit may include a built-in battery.

A further aspect of the disclosure pertains to a medication delivery detector including a body having a proximal end and a distal end. A first connector is coupled to the proximal end of the body, and a second connector is coupled to the distal end of the body. A lumen is formed in the interior of the body, and a plurality of sensors exposed to the lumen serve to detect an identity and concentration of a fluid substance flowing through the lumen, wherein each of the plurality of sensors comprises a receptor and sensor electrodes. A valve is integrated into the lumen of the body, such that the fluid substance flows through the valve.

In some embodiments, at least one flow rate sensor is provided. The valve may also be integrated into the body between the at least one flow rate sensor and the second connector on the distal end.

Still a further aspect of this disclosure relates to a method for detecting a fluid substance of a target medication. The method comprises connecting a medication detector to a catheter using a first connector and connecting the medication detector to an infusion device using a second connector. The method further comprises delivering the fluid substance via the infusion device to the medication detector, transporting the fluid substance through a lumen formed in a body of the medication detector, detecting an identity and concentration of the fluid substance using at least one sensor exposed to the lumen, and actuating a valve between an open position and a closed position to control a flow of the fluid substance through the lumen and into the catheter for delivery to a patient. The method may further include actuating the valve to the closed position when the fluid substance detected by the at least one sensor is not the target medication.

### BRIEF DESCRIPTION OF THE DRAWINGS

A more particular description of the present disclosure will be rendered by reference to specific embodiments thereof that are illustrated in the appended drawings. It is appreciated that these drawings depict only typical embodiments of the invention and are therefore not to be considered limiting of its scope. Example embodiments of the invention will be described and explained with additional specificity and detail through the use of the accompanying drawings in which:

FIG. 1 is a cross-sectional view of a medication delivery detector, according to one or more embodiments shown and described herein;

FIG. 2 is a cross-sectional view of the medication delivery detector of FIG. 1, further including a valve, according to one or more embodiments shown and described herein;

FIG. 3 is a cross-sectional view of the medication delivery detector having a plurality of sensors, according to one or more embodiments shown and described herein;

FIG. 4 is a cross-sectional view of the medication delivery detector having the plurality of sensors, and further including a battery, according to one or more embodiments shown and described herein;

FIG. 5 is a cross-sectional view of the medication delivery detector having the plurality of sensors and the valve, and further including at least one flow rate sensor, according to one or more embodiments shown and described herein; and

FIG. 6 is a cross-sectional view of the medication delivery detector having the plurality of sensors, the valve, and the at least one flow rate sensor, according to one or more embodiments shown and described herein.

Other features and advantages of the present disclosure will be understood from the following embodiments described in detail herein and with reference to the accompanying drawings, in which like reference numerals represent the same or similar components.

### DETAILED DESCRIPTION

The present disclosure is directed to a medication delivery detector and a method for detecting a fluid substance of a target medication. In the embodiments described herein, the medication delivery detector includes a body having a proximal end and a distal end, a first connector coupled to the proximal end of the body, a second connector coupled to the distal end of the body, and a lumen formed in the interior of the body. At least one sensor is exposed to the lumen to detect an identity and concentration of a fluid substance flowing through the lumen, wherein the at least one sensor comprises a receptor and sensor electrodes. By detecting the identity and concentration of fluid flowing through the lumen, the medication delivery detector described herein may improve the efficiency and accuracy of medication administration, thereby reducing the risk of error and contributing to better patient outcomes.

Although the present disclosure is directed towards the medication delivery detector described herein, it is to be understood that the present disclosure may assume various alternative variations and step sequences, except where expressly specified to the contrary. It is also to be understood that the specific devices and processes illustrated in the attached drawings, and described in the following specification, are simply exemplary and non-limiting embodiments or aspects. Hence, specific dimensions and other physical characteristics related to the embodiments or aspects disclosed herein are not to be considered as limiting. No aspect, component, element, structure, act, step, function, instruction, and/or the like used herein should be construed as critical or essential unless explicitly described as such.

Also, as used herein, the articles “a” and “an” are intended to include one or more items, and may be used interchangeably with “one or more” and “at least one.” Where only one item is intended, the term “one” or similar language is used. Also, as used herein, the terms “has,” “have,” “having,” or the like are intended to be open-ended terms. Further, the phrase “based on” is intended to mean “based at least partially on” unless explicitly stated otherwise.

As used herein, the terms “communication” and “communicate” refer to the receipt or transfer of one or more signals, messages, commands, or other type of data. For one unit (e.g., any device, system, or component thereof) to be in communication with another unit means that the one unit is able to directly or indirectly receive data from and/or transmit data to the other unit. This may refer to a direct or indirect connection that is wired and/or wireless in nature. Additionally, two units may be in communication with each other even though the data transmitted may be modified, processed, relayed, and/or routed between the first and second unit. For example, a first unit may be in communication with a second unit even though the first unit passively receives data and does not actively transmit data to the second unit. As another example, a first unit may be in communication with a second unit if an intermediary unit processes data from one unit and transmits processed data to the second unit. It will be appreciated that numerous other arrangements are possible.

Some non-limiting embodiments or aspects are described herein in connection with thresholds. As used herein, satisfying a threshold may refer to a value being greater than the threshold, more than the threshold, higher than the threshold, greater than or equal to the threshold, less than the threshold, fewer than the threshold, lower than the threshold, less than or equal to the threshold, equal to the threshold, etc.

“Comprise”, “comprising”, and “comprises” and “comprised of” as used herein are synonymous with “include”, “including”, “includes” or “contain”, “containing”, “contains” and are inclusive or open-ended terms that specifies the presence of what follows e.g. component and do not exclude or preclude the presence of additional, non-recited components, features, element, members, steps, known in the art or disclosed therein.

Embodiments of medication delivery detectors will now be described in additional detail herein. The following will now describe these lubrication systems in detail with reference to the drawings and where like numbers refer to like structures.

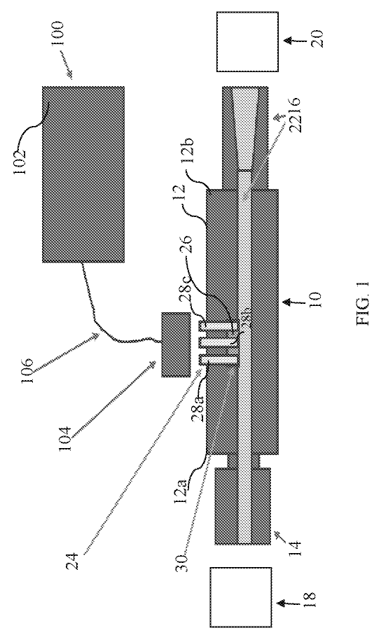

Reference is now made to FIG. 1, which illustrates a medication delivery detector 10. The medication delivery detector 10 may include a body 12 having a proximal end 12a and a distal end 12b (with the term “end” considered to mean “end portion”). The proximal end 12a of the body 12 may be coupled with a first connector 14, such as a male luer, and the distal end 12b of the body 12 may be coupled with a second connector 16, such as a female luer. The first connector 14 may be configured to interface with a catheter 18 or similar device. Likewise, the second connector 16 may be configured to interface with an infusion device 20, such as an infusion line, pump, or syringe. In the embodiments described herein, the first connector 14 and the second connector 16 may each be detachably connected to the catheter 18, the infusion device 20, or another exterior device.

Referring still to FIG. 1, a lumen 22 may be formed in the interior of the body 12, extending between the first connector 14 and the second connector 16. The body 12 may further include at least one sensor 24 designed to detect the presence and concentration of a fluid substance flowing through the lumen 22. The at least one sensor 24 may comprise at least one reference electrode 28a, at least one counter electrode 28b, and at least one sensing electrode 28c. It should be appreciated that, in embodiments in which a surface of the at least one sensor 24 is exposed to the fluid in the lumen, the at least one sensing electrode may be conjugated with at least one electrochemical receptor. Accordingly, the at least one sensor 24 may allow for fluid contact with the fluid substance that enters the infusion device 20 as the fluid flows through the lumen 22 prior to exiting the lumen via the catheter 18.

In the embodiments described herein, it should be further understood that the fluid substance may comprise at least one medication, with the at least one sensor 24 being configured to determine and/or recognize a property of the fluid substance, such as a chemical composition. The at least one medication detected by the at least one sensor 24 may correspond to a target medication intended to be administered to a patient, as will be described in additional detail herein.

Referring still to FIG. 1, the at least one sensor 24 may comprise a redox active receptor positioned at the exposed surface of the sensing electrode 28a. The redox active receptor may use aptamer, protein or antibody receptors selected to bind to chemical groups or recognition elements on a target specifies. In these embodiments, the receptor may detect the presence and/or concentration of a target species in the fluid substance via a change in electron transfer from the receptor to the electrode of the at least one sensor 24.

For example, in embodiments in which the at least one sensor includes an aptamer receptor, the aptamer may form a three-dimensional shape when coming into contact with the target species of the fluid substance. Specifically, in an unbound state, the aptamer may be conjugated with one or more redox reporters, which may be configured to transfer electrons at a predetermined and/or known rate to the sensor electrodes 28a. In the presence of the target species, the aptamer may change conformation, thereby adjusting the positioning of the one or more redox reporters such that the one or more redox reporters move closer or further from the surface of the sensor electrode 28a. Accordingly, it should be appreciated that the receptor of the at least one sensor 24 may experience a change in shape when a target medication is detected, resulting in a measurable change in electrical signal that is proportional to the concentration of the target medication.

Referring still to FIG. 1, the at least one sensor 24 may also be programmed to respond when the target medication concentration is within a predetermined threshold. For example, when the target medication binds to the aptamer, the electrical signal generated may be measured by a component of a control unit 100, particularly a potentiostat 102. Accordingly, the at least one sensor 24 selected for the medication delivery detector 10 can be selected according to the target species needing to be monitored. The binding of the aptamer or other receptors to the target species of the fluid substance flowing through the lumen 22 allows for real-time detection of medication without the need for reagents or sample preparations.

As further depicted in FIG. 1, the at least one sensor 24 may be further coupled to a power connector 104 to allow for communication between the medication delivery detector 10 and the control unit 100 via a cable 106 connecting the power connector 104 to the control unit 100. The control unit 100 may further include the potentiostat 102, which may be programmed to apply potential waveforms and measure electrical currents resulting from the change in electrical signal produced by the at least one sensor 24.

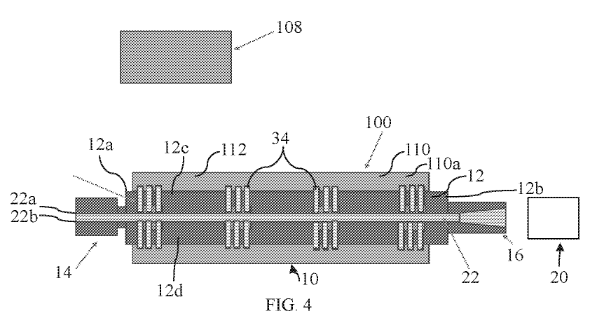

Referring now to FIGS. 1 and 4, it should be further appreciated that the control unit 100 may be battery powered or connected to a power supply. In these embodiments, the control unit 100 may further include a display unit 108, as most clearly illustrated in FIG. 4. Upon detecting the change in the electrical signal produced by the at least one sensor 24, the control unit 100 may communicate (e.g., via Wi-Fi or any other similar communication channel) with the display unit 108 to display the associated medication’s identity and concentration.

Communication between the control unit 100 and the display unit 108 may be further used to transmit information between various components of the medication delivery detector 10. For example, the change in the electrical signal generated by a change in conformation of the at least one sensor 24 may be reported to the patient’s electronic medical record (EMR) and/or the infusion device 20. The presence of the at least one medication and the concentration of the at least one medication detected by the at least one sensor 24 and recorded either manually, based on information displayed on the display unit 108, or electronically (e.g., via Wi-Fi, etc.), in the patient’s EMR, at which point the at least one medication may be compared with the target medication.

In the event that the at least one medication being administered and the target medication do not match, the control unit 100 may be trigger an alarm device (not pictured). The alarm device may alert clinicians in the form of beeping, flashing, vibrating, or similar means. The alarm device’s ability to signal an error in real-time allows for prompt corrective action.

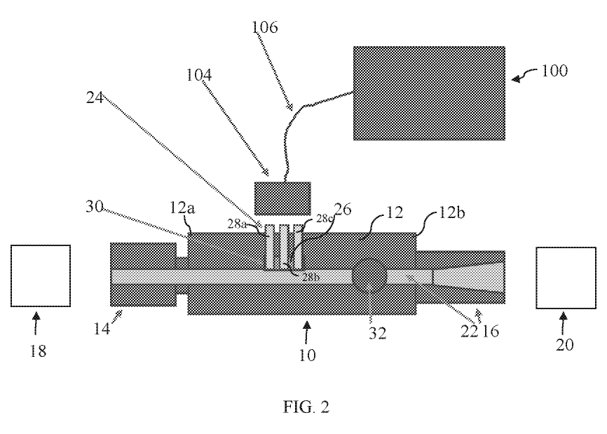

Referring now to FIG. 2, the medication delivery detector 10 may further include a valve 32, such as a solenoid valve, integrated into the body 12 of the medication delivery detector 10. The valve 32 may be configured to actuate between an open position and a closed position. In the open position, the valve 32 permits passage of the at least one medication through the lumen 22. In the instance where the at least one medication detected by the at least one sensor 24 is not the target medication, the valve 32 will be actuated to the closed position to prevent further passage of the at least one medication from the infusion device 20 into the body 12, thus cutting off infusion into the patient.

In these embodiments, the control unit 100 may be communicatively coupled to the valve 32 to control actuation of the valve 32 according to the response of the receptor 26 and cross-checking of the patient’s EMR. For example, the medication delivery detector 10 may be designed to detect medication A and medication B. If medication A is the target medication, and medication B is the at least one medication flowing through the lumen 22, the at least one sensor 24 programmed to detect medication A will not experience the change in the electrical signal. Rather, the at least one sensor 24 programmed to detect medication B will experience the change in electrical signal. Accordingly, the control unit 100 may decipher that the change in the electrical signal is associated with medication B, and when compared against the EMR which identifies medication A as the target medication, the control unit 100 may actuate the valve 32 to the closed position to prevent further infusion of medication B. In these embodiments, the alarm device described herein may also be activated to alert clinicians when the type and the amount of medication detected does not match the target medication intended to be infused. Alternatively, if the at least one sensor 24 programmed to detect medication A is responsive, the valve 32 may remain in the open position, as the response from the at least one sensor 24 programmed to detect medication A indicates that medication A matches the target medication.

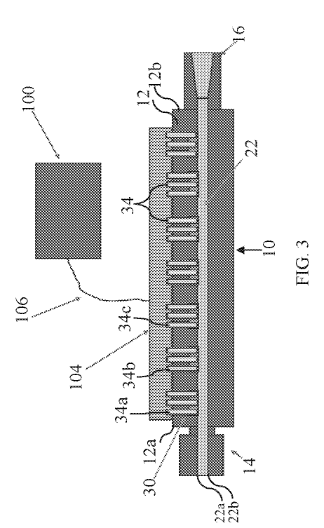

Turning now to FIG. 3, in some embodiments, a plurality of sensors 34 may be disposed throughout the body 12 between the first connector 14 and the second connector 16. Each of the plurality of sensors 34 may be equally or variably spaced in relation to another. The power connector 104 may be coupled to each of the plurality of sensors 34 along a first side 22a of the lumen 22. As each of the plurality of sensors 34 is designed to detect a different medication, a first sensor 34a may be configured to detect a first medication, a second sensor 34b may be configured to detect a second medication, and a third sensor 34c may be configured to detect a third medication, which each of the first medication, the second medication, and the third medication being different from one another. Furthermore, although FIG. 3 depicts the plurality of sensors 34 as including three sensors, it should be appreciated that the plurality of sensors 34 may include any number of sensors (corresponding to any number of different medications) without departing from the scope of the present disclosure. In these embodiments, the use of the plurality of sensors 34, rather than a single sensor, may enable time and cost savings by allowing the same medication delivery detector 10 to be used continuously as the patient receives a series of medications. Furthermore, because the aptamer-based sensor 30 is capable of binding to the variety of target molecules, additional sensors (not pictured) may be selected and integrated into the medication delivery detector 10 to detect other fluid substance-targets.

In the embodiments described herein, it should be understood that each of the plurality of sensors 34 may be particularly configured to detect common medications that lead to medication errors such as morphine, potassium chloride, heparin, vancomycin, cefazolin, acetaminophen, etc. Similarly, in other embodiments, each of the plurality of sensors 34 may be configured to detect common cancer medications such as cyclophosphamide, methotrexate, doxorubicin, paclitaxel, trastuzumab, etc. However, as noted hereinabove, the plurality of sensors 34 may be configured to detect any type of medication without departing from the scope of the present disclosure.

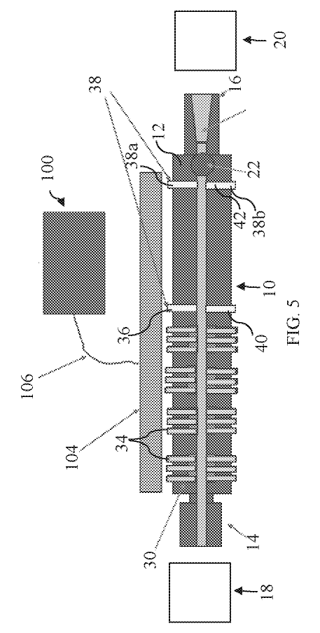

Turning now to FIGS. 4 - 6, additional embodiments of the medication delivery detector 10 are depicted, in which the plurality of sensors 34 are disposed throughout the body 12 on the first side 22a of the lumen 22 and a second side 22b of the lumen 22. For example, as shown in FIGS. 4 and 5, the plurality of sensors 34 may be positioned circumferentially about the lumen 22 of the medication delivery detector 10, such that at least a portion of each of the plurality of sensors 34 is disposed on the first side 22a of the lumen 22 and on the second side 22b of the lumen 22. In these embodiments, the plurality of sensors 34 may be in the form of rings, or any other similar shape configured to extend circumferentially about the lumen 22 of the medication delivery detector 10.

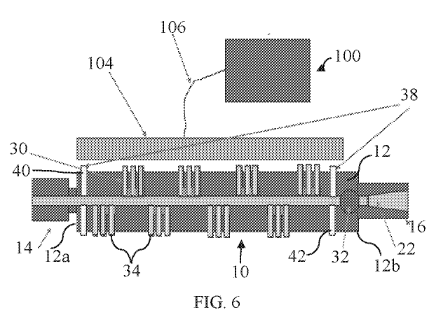

In other embodiments, as shown in FIG. 6, the plurality of sensors 34 may include separate sensors which are positioned on the first side 22a of the lumen 22 and the second side 22b of the lumen 22, respectively. In these embodiments, the plurality of sensors 34 may be staggered, such that each of the plurality of sensors 34 on the first side 22a of the lumen 22 do not align with each of the plurality of sensors 34 on the second side 22b of the lumen 22.

Referring again to FIG. 4, the control unit 100 may further comprise a battery control unit 110, which may include a built-in battery 112 to serve as a power source for the medication delivery detector 10. The battery control unit 110 may be coupled directly to the body 12, thus making the medication delivery detector 10 self-contained and cordless. The battery control unit 110 may be adapted to communicate with the patient EMR system, the infusion device 20, and the display unit 108. In these embodiments, a battery control unit 110a may be coupled to a first body side 12c to communicate with the plurality of sensors 34 on the first side of the lumen 22a, and may be further coupled to a second body side 12d to communicate with the plurality of sensors 34 on the second side of the lumen 22b. It should be appreciated that the battery control unit 110a may extend along the length of the body 12, between the first connector 14 and the second connector 16.

Turning now to FIG. 5, in some embodiments, the medication delivery detector 10 may further include at least one flow rate monitoring device 36, such as at least one flow rate sensor 38, positioned within the body 12 along the lumen 22. In these embodiments, a first portion 38a of the at least one flow rate sensor 38 may extend outwardly from the first side of the lumen 22a, and a second portion 38b of the at least one flow rate sensor 38 may extend outwardly from the second side of the lumen 22b. As illustrated in FIG. 5, each of the flow rate sensor components 38a and 38b may be piezoelectric transducers that emit and detect ultrasound pulses at fixed interval. In these embodiments, the Doppler shift in the ultrasound pulse in the presence of flow may be measured, and the flow presence may then be correlated to a flow velocity.

The variety of possible arrangements available for placement of the plurality of sensors 34 along the body 12 allows for a variety of positions for the at least one flow rate sensor 38 along the body 12. For example, the at least one flow rate sensor 38 may be located between the plurality of sensors 34 and the valve 32. In other embodiments, the plurality of sensors 34 may be positioned between the first connector 14 and a first flow rate sensor 40, and a second flow rate sensor 42 may be located between the first flow rate sensor 40 and the valve 32.

Furthermore, the medication delivery detector 10 may be adapted to connect in a serial fashion to other medication delivery systems (not shown). Additional sensors, such as a temperature sensor (not shown), may be integrated into the body 12 of the medication delivery detector 10 in these embodiments.

Referring now to FIG. 6, another embodiment of the medication delivery detector 10 include the at least one flow rate sensor 38 is depicted. In this embodiment, the plurality of sensors 34 may be disposed throughout the body 12 between the first flow rate sensor 40 on the proximal end 12a and the second flow rate sensor 42 on the distal end 12b of the body 12. The first flow rate sensor 40 may be in between the first connector 14 and the plurality of sensors 34, and the second flow rate sensor 42 may be between the plurality of sensors 34 and the valve 32. It should be appreciated that the arrangement depicted in FIG. 6 provides a more compact medication delivery detector 10, thus reducing the overall size of the medication delivery detector 10.

In another embodiment, in addition to the receptor, sensors may be included to detect other fluid properties, such as pH, ionic strength, or temperature.

Summarizing, this disclosure relates to any one or more of the following items in any ordered combination:

1. A medication delivery detector, comprising:

a body having a proximal end and a distal end;

a first connector coupled to the proximal end of the body;

a second connector coupled to the distal end of the body;

a lumen formed in the interior of the body; and

at least one sensor exposed to the lumen to detect an identity and concentration of a fluid substance flowing through the lumen, wherein the at least one sensor comprises a receptor and sensor electrodes.

2. The medication delivery detector of item 1, further including a valve integrated into the lumen of the body, wherein the fluid substance flows through the valve.

3. The medication delivery detector of item 2, wherein the valve alternates between an open position and a closed position.

4. The medication delivery detector of item 2, wherein the valve is between the at least one sensor and the second connector.

5. The medication delivery detector of any of items 1-4, wherein the at least one sensor comprises a redox active receptor.

6. The medication delivery detector of any of items 1-5, wherein the at least one sensor includes a plurality of sensors, and the plurality of sensors are spaced along a length of the body.

7. The medication delivery detector of item 6, wherein the plurality of sensors disposed throughout the body extend circumferentially about the lumen formed in the interior of the body.

8. The medication delivery detector of item 6, wherein the plurality of sensors disposed throughout the body on a first side of the lumen are staggered in relation to the plurality of sensors on a second side of the lumen.

9. The medication delivery detector of any of items 1-8, wherein at least one flow rate sensor is integrated into the body.

10. The medication delivery detector of item 9, wherein the at least one sensor is integrated into the body between a first flow rate sensor on the proximal end and a second flow rate sensor on the distal end.

11. The medication delivery detector of item 9, wherein the at least one flow rate sensor is integrated into the body between the at least one sensor and a valve.

12. The medication delivery detector of item 9, wherein a valve is integrated into the body between the at least one flow rate sensor and the second connector on the distal end.

13. The medication delivery detector of any of items 1-12, configured to interface with a catheter.

14. The medication delivery detector of any of items 1-13, configured to interface with an infusion device.

15. The medication delivery detector of any of items 1-14, further comprising a control unit coupled to the body, wherein the control unit includes a built-in battery.

16. A medication delivery detector, comprising:

a body having a proximal end and a distal end;

a first connector coupled to the proximal end of the body;

a second connector coupled to the distal end of the body;

a lumen formed in the interior of the body;

a plurality of sensors exposed to the lumen to detect an identity and concentration of a fluid substance flowing through the lumen, wherein each of the plurality of sensors comprises a receptor and sensor electrodes; and

a valve integrated into the lumen of the body, wherein the fluid substance flows through the valve.

17. The medication delivery detector of item 16, further including at least one flow rate sensor.

18. The medication delivery detector of item 17, wherein the valve is integrated into the body between the at least one flow rate sensor and the second connector on the distal end.

19. A method for detecting a fluid substance of a target medication, comprising:

connecting a medication detector to a catheter using a first connector and connecting the medication detector to an infusion device using a second connector;

delivering the fluid substance via the infusion device to the medication detector;

transporting the fluid substance through a lumen formed in a body of the medication detector;

detecting an identity and concentration of the fluid substance using at least one sensor exposed to the lumen; and

actuating a valve between an open position and a closed position to control a flow of the fluid substance through the lumen and into the catheter for delivery to a patient.

20. The method of item 19, further comprising:

actuating the valve to the closed position when the fluid substance detected by the at least one sensor is not the target medication.

Although the inventions are described in conjunction with specific embodiments, many alternatives, modifications, and variations will be apparent to those skilled in the art. Accordingly, it embraces all such alternatives, modifications, and variations that fall within the spirit and scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, or identification of any reference in this application shall not be construed as an admission that such reference is available as prior art to the present disclosure.

What is claimed is:

1. A medication delivery detector, comprising:
a body having a proximal end and a distal end;
a first connector coupled to the proximal end of the body;
a second connector coupled to the distal end of the body;
a lumen formed in the interior of the body; and
at least one sensor exposed to the lumen to detect an identity and concentration of a fluid substance flowing through the lumen, wherein the at least one sensor comprises a receptor and sensor electrodes.

2. The medication delivery detector of claim 1, further including a valve integrated into the lumen of the body, wherein the fluid substance flows through the valve.

3. The medication delivery detector of claim 2, wherein the valve alternates between an open position and a closed position.

4. The medication delivery detector of claim 2, wherein the valve is between the at least one sensor and the second connector.

5. The medication delivery detector of claim 1, wherein the at least one sensor comprises a redox active receptor.

6. The medication delivery detector of claim 1, wherein the at least one sensor includes a plurality of sensors, and the plurality of sensors are spaced along a length of the body.

7. The medication delivery detector of claim 6, wherein the plurality of sensors disposed throughout the body extend circumferentially about the lumen formed in the interior of the body.

8. The medication delivery detector of claim 6, wherein the plurality of sensors disposed throughout the body on a first side of the lumen are staggered in relation to the plurality of sensors on a second side of the lumen.

9. The medication delivery detector of claim 1, wherein at least one flow rate sensor is integrated into the body.

10. The medication delivery detector of claim 9, wherein the at least one sensor is integrated into the body between a first flow rate sensor on the proximal end and a second flow rate sensor on the distal end.

11. The medication delivery detector of claim 9, wherein the at least one flow rate sensor is integrated into the body between the at least one sensor and a valve.

12. The medication delivery detector of claim 9, wherein a valve is integrated into the body between the at least one flow rate sensor and the second connector on the distal end.

13. The medication delivery detector of claim 1, configured to interface with a catheter.

14. The medication delivery detector of claim 1, configured to interface with an infusion device.

15. The medication delivery detector of claim 1, further comprising a control unit coupled to the body, wherein the control unit includes a built-in battery.

16. A medication delivery detector, comprising:
a body having a proximal end and a distal end;
a first connector coupled to the proximal end of the body;
a second connector coupled to the distal end of the body;
a lumen formed in the interior of the body;
a plurality of sensors exposed to the lumen to detect an identity and concentration of a fluid substance flowing through the lumen, wherein each of the plurality of sensors comprises a receptor and sensor electrodes; and
a valve integrated into the lumen of the body, wherein the fluid substance flows through the valve.

17. The medication delivery detector of claim 16, further including at least one flow rate sensor.

18. The medication delivery detector of claim 17, wherein the valve is integrated into the body between the at least one flow rate sensor and the second connector on the distal end.

19. A method for detecting a fluid substance of a target medication, comprising:
connecting a medication detector to a catheter using a first connector and connecting the medication detector to an infusion device using a second connector;
delivering the fluid substance via the infusion device to the medication detector;
transporting the fluid substance through a lumen formed in a body of the medication detector;
detecting an identity and concentration of the fluid substance using at least one sensor exposed to the lumen; and
actuating a valve between an open position and a closed position to control a flow of the fluid substance through the lumen and into the catheter for delivery to a patient.

20. The method of claim 19, further comprising:
actuating the valve to the closed position when the fluid substance detected by the at least one sensor is not the target medication.
